(19) United States
(12) Patent Application Publication
(10) Pub. No.: US 2006/0233900 A1
(43) Pub. Date: Oct. 19, 2006

(54) Preparation for reducing the appetite, producing a satiated feeling and/or for weight loss in children

(76) Inventor: Günther Beisel, Monheim (DE)

Correspondence Address: FRIEDRICH KUEFFNER, 317 MADISON AVENUE, SUITE 910, NEW YORK, NY, 10017, US

(21) Appl. No.: 10/539,227

(22) PCT Filed: Dec. 8, 2003
(86) PCT No.: PCT/EP03/13902

§ 371 Date: May 15, 2006

(30) Foreign Application Priority Data
Dec. 19, 2002 (DE) 20219667.4
Dec. 19, 2002 (DE) 10258993.3

(51) Int. Cl. A61K 36/48

(52) U.S. Cl. 424/757

(57) ABSTRACT

The present invention relates to a preparation for children at the age of up to 17 years for long-lasting appetite reduction, satiation and/or weight reduction, comprising carob bean flour or a mixture of these substances and at least one fatty acid and/or its derivatives.

The present invention relates to a preparation for appetite reduction, satiation and/or weight reduction, which is suitable in particular for children at the age of up to 17 years.

Numerous experiments have been undertaken to break down superfluous accumulations of fat in the human body medicinally or to prevent their formation. There are, for example, “appetite suppressants”, which attempt biochemically to suggest to the body an aversion to food intake. These compositions in some cases have appreciable harmful side effects.

In addition to the numerous known diet proposals, there are also mechanical and electromechanical agents, by which a specific breakdown of fat or building up of muscle should take place. The action of such agents, however, is very doubtful.

DE 4025912 discloses a composition for oral consumption, which consists of a container dissolvable in the stomach and releasing the contents. This is filled with a substance which, after its release in the stomach, increases its volume and thereby suggests a feeling of satiation to the body. The disadvantage of this satiating agent is the that the danger of intestinal occlusions exists.

Furthermore, DE 199 42 417 discloses spongy preparations containing stably crosslinked compounds, which increase their volume in the stomach and thus produce a feeling of satiation. However, the production of these preparations necessitates additional process steps for the introduction of stable crosslinkages.

On account of continuously increasing health consciousness, however, a further improvement in compositions for producing a satiation effect is of high medical and economic relevance.

The administration of the described satiating agents in children is problematical. In particular, visually and as regards taste there is low acceptance.

It is the object of the present invention to make available a composition which rapidly suppresses the feeling of hunger and contributes to appetite reduction and/or weight reduction. At the same time, the composition should be simple to administer, furthermore digestible and not overburdening for the body, and suitable for children preferably at the age of up to 17 years.

This object is achieved by means of a preparation of a swelling substance for long-lasting appetite reduction, satiation and/or weight reduction, comprising carob bean flour and at least one fatty acid and/or its derivatives.

The carob bean flour employed according to the invention is obtained from the fruits of the carob bean tree (Caesalpiniaceae). The product, which is also called carobin, carubin or karobbe swells in cold water and gives more viscous solutions than many types of tragacanth, but does not form any gel. It is known to employ carob bean flour as a thickening agent for foodstuffs, chewing gum and tobacco goods. In particular it is also known to employ the ground carob bean as a coffee and cocoa substitute for chocolate. In particular, it is also suitable for dietetic foodstuffs, since it does not have the known disadvantageous health effects of chocolate. Since, on the other hand, it has a similar taste to chocolate or cocoa, it is particularly suitable in combination with the fatty acids employed according to the invention for children who ought to reduce their weight.

Carob bean flour is contained in the preparation according to the invention in amounts from 3% by weight to 20% by weight, particularly preferably 30% by weight to 80% by weight, very particularly preferably 35% by weight to 70% by weight.

The composition according to the invention contains at least one fatty acid. By this means, in particular, the digestion of food is slowed. The result is a longer residence time in the stomach, which in turn causes a longer-lasting satiation effect.

According to the invention, various fatty acids can be used. Fatty acids having a number of at least 6 carbon atoms in the molecule, preferably having a chain length of C₈-C₂₂, particularly preferably having a chain length of C₁₂-C₁₈, are particularly suitable. Fatty acids in saturated or unsaturated form and also derivatives and/or salts of these fatty acids are further included according to the invention here. The present invention also includes food fatty acids, their derivatives and/or salts.

Derivatives are to be understood according to the invention as meaning esters of the fatty acids with glycerol or esters and ethers of the fatty acids with alcohols having a number of at least 2 carbon atoms, preferably a chain length of C₂ to C₂₂.

Examples of additives according to the invention are lauric acid, myristic acid, palmitic acid, stearic acid or oleic acid. This list, however, is non-limiting for the present invention. Derivatives of the fatty acids according to the invention can also be corresponding alkali metal or ammonium salts of these fatty acids. In variants of the present preparation, the fatty acids can be present in chemically bound form in lecithin and are released by enzymatic processes during the chewing/sucking or swallowing process. The fatty acids can be incorporated in the preparation according to the invention in finely disperse form by trituration with colloidal silicic acid. The preparation according to the invention advantageously contains fatty acids in a proportion from 0.7 to 70 mg/g, preferably from 2.5 to 50 mg/g and particularly preferably from 10 to 20 mg/g, of the basic composition.

The present invention furthermore relates to preparations of the type mentioned beforehand, which additionally to said fatty acids, their derivatives and/or salts contain chitosan and its derivatives and/or salts. For the chitosan contained in the preparation according to the invention, proportions from 1 to 150 mg/g, preferably 20 to 120 mg/g and particularly preferably 60 to 80 mg/g, of basic composition are advantageous. Provided a chewing gum weighs 5 g, this corresponds to 5 to 750 mg of chitosan per chewing gum, preferably 100 to 600 mg/chewing gum and particularly preferably 300 to 400 mg/chewing gum.

Furthermore, various synthetic, semisynthetic or natural polymers can be contained in the composition according to the invention.

Suitable synthetic polymers are, for example, polyurethanes, polyacrylates, polymethacrylic acid esters, homo- and copolymers of vinyl acetate.

In one embodiment of the present invention organic polymers can be employed. Organic polymers can, with respect to their origin or synthesis, be natural, semisynthetic or synthetic. Examples of suitable polymers are polyurethanes, polyacrylates, poly(meth)acrylic acid esters, homo- and copolymers of vinyl acetate, polyvinyl acetate, polyacrylic acid, polyethylene glycol, polyvinylpyrrolidone, cellulose, ethers, diethylcellulose or cellulose esters, such as cellulose diacetate, cellulose triacetate, cellulose acetate propionate and cellulose acetate, methylcellulose, hydroxypropylcellulose, hydroxypropylmethylcellulose, or sodium carboxymethylcellulose (preferably those compounds having relatively high viscosity); butyrate, low-substituted hydroxypropylcellulose, carboxymethylcellulose (having 2- or 3-valent cations), Na starch glycolate, glycosaminoglycans such as chondroitin sulfate or hyaluronic acid, collagen, albumin, keratins, conchagens, fibroin, elastins, chitin.

Furthermore, hydrocolloids, in particular based on polysaccharides, can be employed.

The use of anionic polymers is also conceivable. These preferably include polysaccharides, in particular polyuronic acid-containing polysaccharides. Alginic acids, their derivatives and salts (alginates), excluding the aluminum salts of the alginic acids, are particularly preferred. However, all other uronic acid-containing compounds can also be used according to the invention. According to the invention, the use of cellulose or cellulose derivatives is furthermore preferred. The use of synthetic or semisynthetic cellulose derivatives, such as, for example, carboxymethylcellulose or of polyacrylates, is conceivable.

Cellulose is to be understood as meaning water-insoluble polysaccharides of the empirical composition (C₆H₁₀O₅)ₙ. To put it more precisely, it is an isotactic β-1,4-polyacetal of cellobiose (4-O-β-D-glucopyranosyl-D-glucose).

Chemically modified celluloses are in general defined by means of polymer-analogous reactions as cellulose derivatives. They comprise both products in which hydroxyl hydrogen atoms of the anhydroglucose units of the cellulose are substituted exclusively, for example by means of esterification and/or the etherification reactions, by organic or inorganic groups, and those which are formed with formal replacement of hydroxyl groups of the natural polymers by functional groups which are not bonded via an oxygen atom (e.g. deoxycelluloses) or by means of intramolecular elimination of water (anhydrocelluloses, celluloses) or oxidation reactions (aldehyde-, keto- and carboxycelluloses). Products which are obtained on cleavage of the C₂, C₃ carbon bond of the anhydroglucose units (dialdehyde- and dicarboxycelluloses), in which the monomer unit characteristic of the cellulose is thus no longer intact, are also numbered among the cellulose derivatives. Cellulose derivatives are also accessible by means of other reactions, e.g. by means of cross-linking or graft copolymerization reactions.

According to the invention, cellulose or cellulose derivatives can be employed as a mixture with pectins. Likewise, mixtures comprising alginic acid or its derivatives, excluding the aluminum salts of alginic acid and pectins, are conceivable.

Alginic acid is a linear polyuronic acid consisting of variable proportions of D-mannuronic acid and L-guluronic acid, which are linked to one another by β-glycosidic bonds, the carboxyl groups not being esterified. A molecule of alginic acid can be composed of approximately 150-1050 uronic acid units, it being possible for the average molecular weight to vary in a range from 30-200 kDa.

The polysaccharide alginic acid ist a constituent of the cell walls of brown algae. The proportion of alginic acid in the dry matter of the algae can here constitute up to 40%. The alginic acid can be obtained by alkaline extraction using methods known per se according to the prior art. The resulting pulverulent alginic acid is thus purely vegetable and has high biocompatibility. It can absorb 300 times the amount of its own weight of water with formation of highly viscous solutions. In the presence of polyvalent cations, alginic acid forms ‘gels’. The formation of alginate gels in the presence of divalent cations, such as calcium or barium, are described in Shapiro I., et al. (Biomaterials, 1997, 18: 583-90). The latter, on account of its toxicity, is not suitable, however, for use in biomedicine. In addition to calcium chloride, calcium gluconate also yields suitable divalent cations. The use of magnesium salts or a mixture of various physiologically acceptable divalent cations is also conceivable.

With respect to the polymers having a low degree of esterification, the use of pectins having a low degree of esterification is also possible according to the invention. Pectins consist of chains of α-1,4-glycosidically linked galacturonic acid units, whose acid groups are 20-80% esterified with methanol. A differentiation is made between highly esterified pectins (>50%) and pectins having a low degree of esterification (<50%). The molar mass varies between 10-500 kDa. The pectins are obtained by acid extraction using methods known per se according to the prior art from the internal constituents of citrus fruit peel, vegetable remains or sugar beet pulp. The resulting pectins (apple pectin, citrus pectin) are thus purely vegetable and have high biocompatibility. They can form gels with absorption of water.

The use of pectin gels in the presence of divalent cations, such as calcium or barium, is also known here. The latter, on account of its toxicity, however, is also not suitable here for use in biomedicine. In addition to calcium chloride, calcium gluconate also yields suitable divalent cations. The use of magnesium salts or a mixture of various physiologically acceptable divalent cations is also conceivable.

Further compounds which can be employed are inorganic substances such as MgCl₂, CaSO₄, Na₂CO₃, CaCO₃, polysilicic acids and clay minerals (such as montmorillonite, zeolites, silicic acids) or organic substances such as mono- and/or disaccharides (mannose, glucose, sucrose, sorbitol), lactose, tartaric acid or urea.

Natural polymers which may finally additionally be mentioned are additionally guar, carob bean flour, konjak flour, starch, pectins, soybean protein, e.g. complete soybean flour, lactoprotein, lupin protein. Moreover, anionic mucilages, e.g. xanthan, tragacanth and insoluble polysaccharides, e.g. chitin, e.g. chitin derivatives are suitable according to the invention. In the process according to the invention, said polymers are initially prepared in a pulverulent embodiment. I.e. any desired powder can be prepared in any desired size. Adsorbates, beadlets, granules, pellets, extrudates and also combinations of these embodiments are included herein. Likewise, use forms are conceivable in which the particles are already coated.

The composition according to the invention can be prepared in various administration forms.

The composition according to the invention contains the compounds described preferably in a pulverulent embodiment. I.e., the composition can be present as an adsorbate, beadlet powder, granules, pellet, extrudate and/or combinations thereof. Likewise, use forms in which the particles are coated are conceivable. The core sizes can be between 50-3000 μm, preferably 100-2000 μm, particularly preferably 700-1500 μm.

The compositions according to the invention preferably present in powder form can be prepared using methods known per se. These include, for example, the preparation of spray formulations; a process and plant for this is, for example, described in EP 0 074 050 B1. Granules can be prepared in a fluidized bed, for example by build-up granulation. The agglomeration takes place in a batch process or in a continuous fluidized bed. The advantage of fluidized bed agglomeration lies in the simultaneous mixing and agglomeration.

The preparation of granules can also be achieved by introducing vehicles and/or spray-dried powder and also, if appropriate, additives into a mixer and producing compact granules by addition of the active components and/or binder and/or additives. Mixers preferably employed in this process are, for example, paddle mixers or plowshare mixers. The liquid components can, for example, be applied drop by drop or sprayed on such that a pasty, sticky phase results. By means of suitable choice of the speed of rotation of the mixing apparatus and/or fast-running knives, the pasty phase is dispersed and compact granules result. Very large lumps are disintegrated by the mixing apparatus and knives and on the other hand fine powders are agglomerated. By addition of enveloping layers can be carried out at a later stage in the mixer at a relatively low speed of rotation of the mixing apparatus and standing knives or in a constructionally related mixer connected at a later stage.

Beside this type of preparation, further process variants are also conceivable. These include, for example, spray drying processes or the preparation of adsorbates in fluidized beds.

Depending on the administration form, the composition according to the invention can be stirred into liquid and drunk or, in the embodiment as coated tablets, tablets or coarse-grain granules, sucked or chewed. The last-mentioned embodiment has the advantage that by chewing and/or sucking the flow of saliva is positively influenced. Owing to the increased flow of saliva, the desired effect of appetite reduction or satiation occurs more rapidly, since a feeling of nutrient absorption is suggested. The additives contained in the preparation are released more rapidly and by means of the chewing/sucking and swallowing the additives prevent gastric emptying or peristalsis of the gastrointestinal tract. In this context, the fatty acids in particular delay gastric emptying and peristalsis. The pectin contained in one process variant according to the invention binds fats and leads in this manner to an effective and gentle cholesterol regulation. Furthermore, the preparation according to the invention leads to an advantageous lowering of the blood sugar.

The preparation according to the invention is thus suitable for appetite reduction, satiation and/or weight reduction. The preparation according to the invention is furthermore suitable for the regulation of the cholesterol metabolism and/or blood sugar level.

The present invention also relates to the use of a preparation of the type according to the invention for the preparation of a composition for appetite reduction, satiation and/or weight reduction. Likewise, the use of the preparation according to the invention for the preparation of a composition for the regulation of the cholesterol metabolism and/or blood sugar level is included.

1. Shear A preparation for children for appetite reduction, satiation and/or weight reduction, comprising carob bean flour and at least one fatty acid and/or its derivatives.

2. The preparation as claimed in claim 1, wherein it contains fatty acids having at least 6 carbon atoms in the molecule, their salts and/or derivatives thereof.

3. The preparation as claimed in claim 1, wherein it contains fatty acids, their derivatives and/or salts having a carbon chain of length from C₈ to C₂₂, preferably from C₁₂ to C₁₈.

4. The preparation as claimed in claim 1, wherein it contains saturated and/or unsaturated fatty acids, their derivatives and/or salts.

5. The preparation as claimed in claim 1, wherein it contains fatty acids, their derivatives and/or salts in a proportion from 0.7 to 70 mg/g, preferably 2.5 to 50 mg/g and particularly preferably 10 to 20 mg/g, of basic composition.

6. The preparation as claimed in claim 1, wherein additionally to the fatty acids, their derivatives and/or salts it contains chitosan, chitosan derivatives and/or its salts.

7. The preparation as claimed in claim 1, wherein it contains pectin, its derivatives and/or salts in a proportion from 0.1 to 10 g, preferably 1 to 5 g and particularly preferably 2 to 4 g, of basic composition.

8. The preparation as claimed in claim 1, wherein it contains 2 g of carob bean flour.

9. The use of a preparation as claimed in claim 1 for long-lasting appetite reduction, satiation and/or weight reduction.

10. The use of the preparation as claimed in claim 1 for the preparation of a composition for long-lasting appetite reduction, satiation and/or weight reduction.
